(19) United States
(12) Patent Application Publication
(10) Pub. No.: US 2026/0248915 A1
(43) Pub. Date: Aug. 27, 2026

(54) NEAR-INFRARED AGGREGATION-INDUCED EMISSION ORGANIC PHOTOTHERMAL MOLECULE AND PREPARATION METHOD THEREOF AND APPLICATION

(71) Applicant: The Chinese University of Hong Kong, Shenzhen, Shenzhen City (CN)

(72) Inventors: Benzhong TANG, Shenzhen City (CN); Zheng ZHAO, Shenzhen City (CN); Haozhe TAN, Shenzhen City (CN); Han YANG, Shenzhen City (CN); Fulong MA, Shenzhen City (CN)

(21) Appl. No.: 19/406,396

(22) Filed: Dec. 2, 2025

(30) Foreign Application Priority Data
Feb. 24, 2025 (CN) 202510199792.8

(51) Int. Cl. A61K 41/00; A61K 9/51; C07D 495/22

(52) U.S. Cl. CPC A61K 41/0052; A61K 9/5169; A61K 9/5192; C07D 495/22

(57) ABSTRACT

A near-infrared emissive aggregation-induced emission organic photothermal molecule and a preparation method thereof and an application are provided. The structure is shown in formula I:

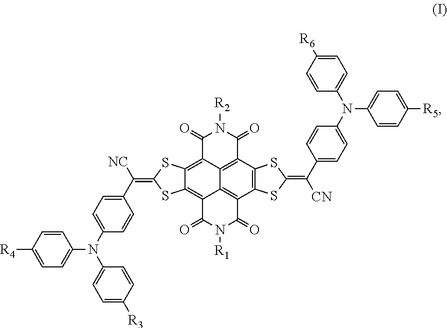

where R₁, R₂, R₃, R₄, R₅, R₆ are independently selected from groups containing alkyl chains. A preparation method of the organic photothermal molecule and an application thereof in preparing near-infrared aggregation-induced emission multifunctional protein nanoparticles are further provided.

### CROSS-REFERENCE TO RELATED APPLICATIONS

This application claims priority to Chinese Patent Application No. 202510199792.8, filed on Feb. 24, 2025, the contents of which are hereby incorporated by reference.

### TECHNICAL FIELD

The present disclosure belongs to the technical field of biomedicine, and in particular relates to a near-infrared emissive aggregation-induced emission organic photothermal molecule and a preparation method thereof and an application.

### BACKGROUND

Glioblastoma multiforme (GBM) is a common and malignant brain tumor that is difficult to treat. Conventional treatments, including surgery, chemotherapy, and radiotherapy, are often ineffective due to significant technical and biological hurdles. For example, surgical resection is complicated and often leads to recurrence, while systemic chemotherapy suffers from drug resistance, poor blood-brain barrier (BBB) permeability, and nonspecific biodistribution. Radiotherapy, although precise, is hampered by intrinsic and acquired radio-resistance in heterogeneous tumor populations. In recent years, near-infrared (NIR) phototheranostic nanomedicines have attracted widespread attention in the early diagnosis and precise treatment of tumors and have achieved significant research progress. The advantages of NIR phototheranostics lie in the excellent NIR imaging effects and efficient photothermal killing ability, making it an important technology for non-invasive tumor treatment. Nevertheless, traversing critical biological barriers, particularly the BBB, remains the primary and significant challenge for nanomedicine-based GBM eradication. Although traditional surfactants nanoengineering strategies that use active ligands, such as apolipoprotein E (ApoE) peptide and Angiopep-2, may improve BBB permeability, they face inherent limitations, including complex preparation and characterization processes, intrinsic biotoxicity associated with surfactants, and variability in efficacy related to ligand-ratios.

Notably, transferrin (Tf), an iron-transport protein, presents a promising solution. It exhibits exceptional biosafety and intrinsic BBB-traversing capability through ligand-receptor interactions with abundantly expressed TfRs on the BBB. Combining transferrin with NIR phototheranostics not only avoids complicated modifications but also enables precise diagnosis and treatment of brain tumors. However, traditional NIR phototheranostic materials, such as indocyanine green (ICG), after binding with proteins, suffer from concentration-dependent aggregation-caused quenching (ACQ), leading to less than ideal image quality. The high signal-to-noise ratio also poses a significant challenge for their use in precise imaging. In addition to photostability stability, issues such as thermal stability, and photobleaching also remain significant hurdles for NIR phototheranostic materials.

Therefore, how to provide NIR phototheranostic materials with excellent performance that may be effectively applied in a transferrin nanomedicine system has become an urgent need to improve the accuracy of GBM diagnosis and treatment.

### SUMMARY

To solve the above technical problems, the present disclosure proposes a near-infrared emissive aggregation-induced emission organic photothermal molecule and a preparation method thereof and an application.

To achieve the above objective, the present disclosure provides the following technical solutions.

A near-infrared emissive aggregation-induced emission organic photothermal molecule, with a structure shown in formula I:

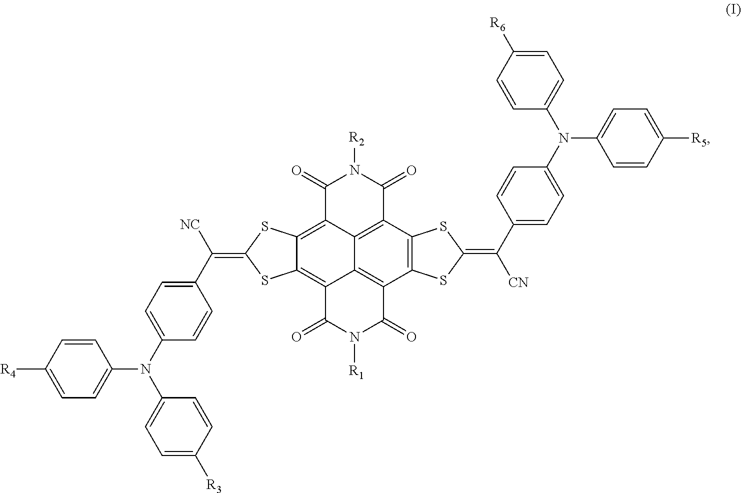

where, R₁, R₂, R₃, R₄, R₅, R₆ are independently selected from groups containing alkyl chains.

In an embodiment, the alkyl chain is one of C₁-C₂₀ straight chain, C₁-C₂₀ branched chain, C₁-C₂₀ cyclic alkyl chain, an alkyl chain with carbon atoms substituted by one or more of oxygen atoms, alkenyl, alkynyl, aryl, carbonyl, hydroxyl, amino, carboxyl, nitro, or ester groups, and an alkyl chain with hydrogen atoms substituted by one or more of fluorine atoms, chlorine atoms, bromine atoms, or and iodine atoms.

In an embodiment, the structure of the near-infrared emissive aggregation-induced emission organic photothermal molecule is as follows:

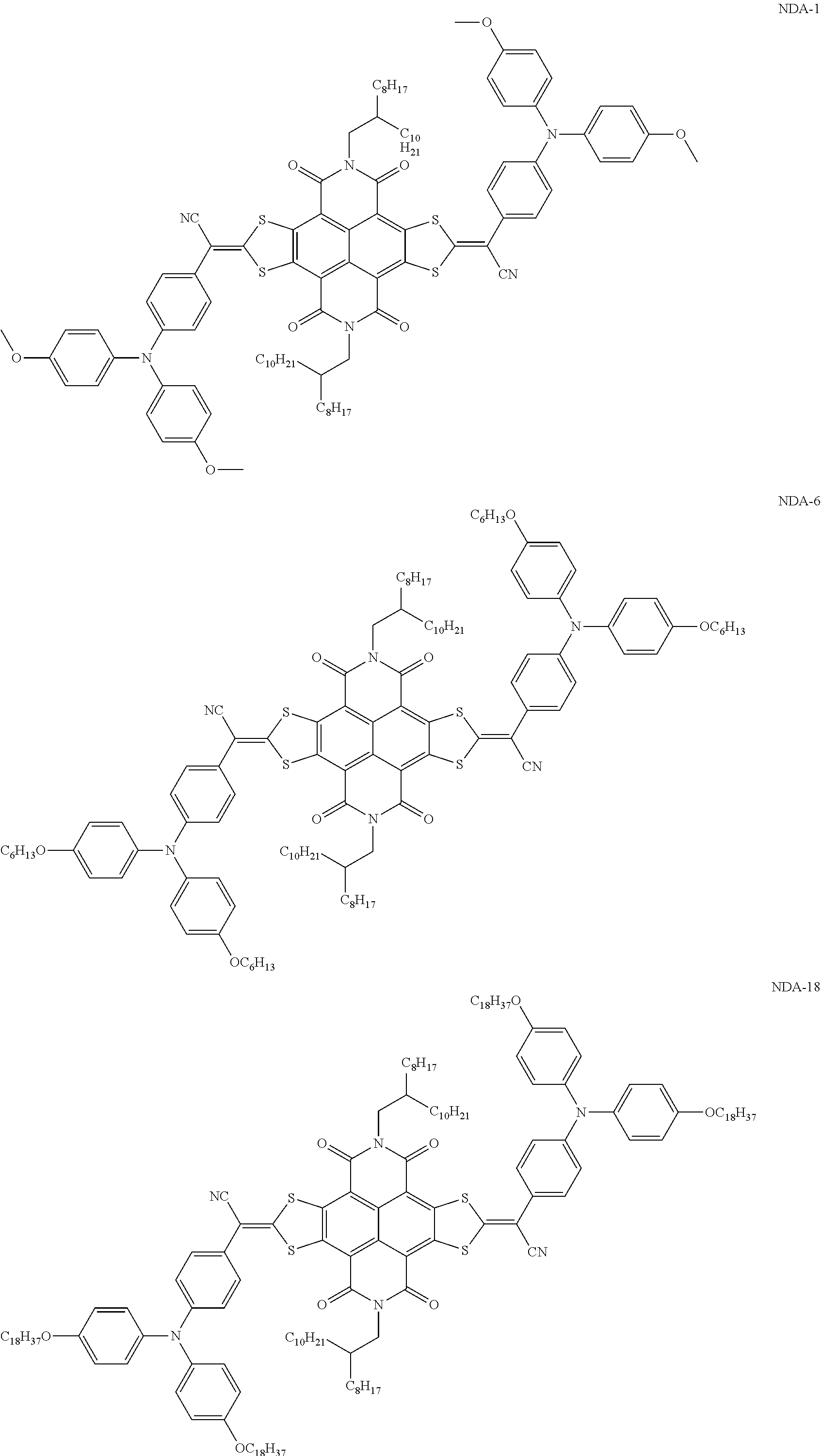

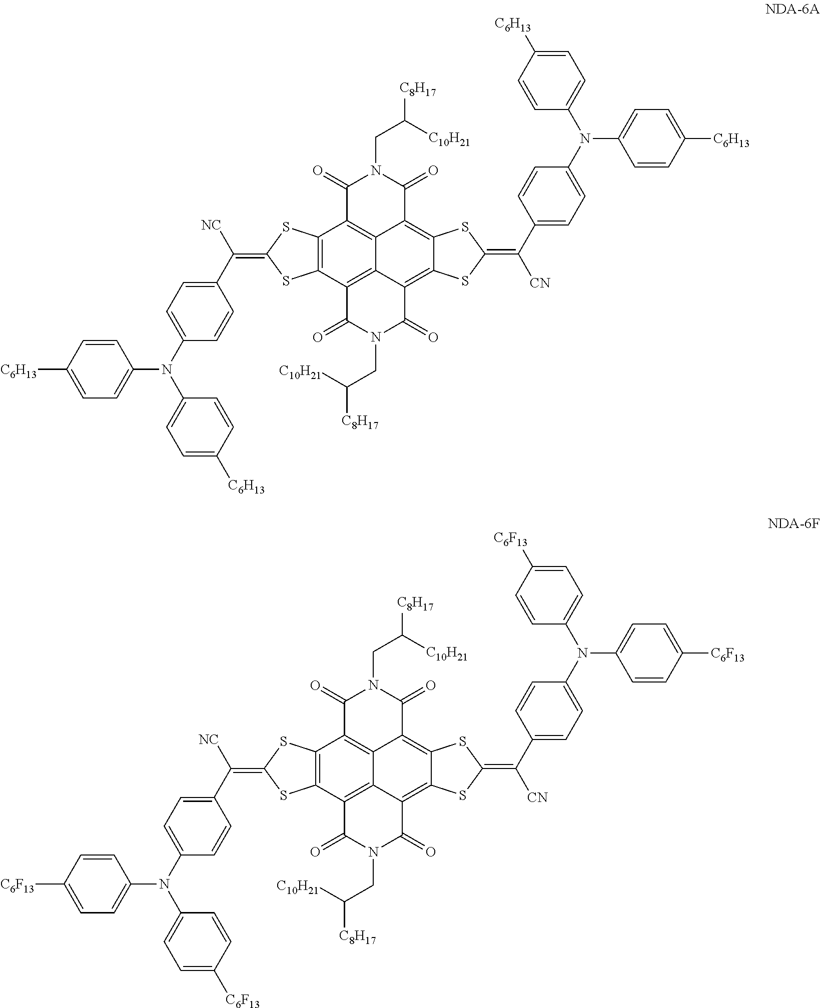

Beneficial effects are as follows. The present disclosure provides a novel near-infrared emissive aggregation-induced emission organic photothermal molecule with an electron donor-electron acceptor structure, using naphthalenediimide-fused 2-(1,3-dithiol-2-ylidene) acetonitrile with long alkyl chains as the electron acceptor group and triphenylamine (TPA) as the molecular rotor and electron donor unit. Under laser irradiation, it may both emit near-infrared-II (NIR-II) light and generate heat, with good photostability. Furthermore, long alkyl chains are also introduced into the triphenylamine to regulate the stacking mode of the molecules in the aggregate state. Specifically, the large conjugated backbone of the electron acceptor group allows for large electron delocalization, forming a lower band gap, with planar structure conducive to enhancing long-wavelength absorption ability and high molar absorptivity. The long alkyl chains on the electron acceptor avoid excessive intermolecular stacking, preventing disadvantages such as poor imaging effects, high signal-to-noise ratio, and poor photostability caused by aggregation-caused quenching. The electron-rich TPA serves as the electron donor in the present disclosure, facilitating an efficient twisted intramolecular charge transfer (TICT) effect. This process thereby enhances both the near-infrared luminescence and its photothermal conversion capability. The long alkyl chains in triphenylamine provide greater rotational freedom for the molecular rotor. In the aggregated state, intermolecular interactions are greatly suppressed, ensuring the efficient and stable photothermal performance. Therefore, the near-infrared emissive aggregation-induced emission organic photothermal molecule provided by the present disclosure may achieve efficient NIR emission performance and excellent photothermal performance, providing material support for the subsequent construction of functional protein nanoparticles.

A preparation method of the near-infrared emissive aggregation-induced emission organic photothermal molecule, including the following steps:
step 1 reacting sodium hydride and 4-nitrophenylacetonitrile under ice bath conditions, then adding carbon disulfide to continue the reaction at room temperature, then adding 4,5,9,10-tetrabromo-2,7-bis(2-octyldodecyl)benzo[lmn][3,8]phenanthroline-1,3,6,8 (2H,7H)-tetraone and continuing to stir for a reaction; after the reaction is complete, performing post-treatment to obtain a compound 1;
step 2 dissolving the compound 1 and ammonium chloride in an organic solvent for reflux reaction, then adding iron powder to continue the reaction; after the reaction is complete, performing post-treatment to obtain a compound 2; and
step 3 dissolving the compound 2, a halogenated aromatic hydrocarbon or halogenated aromatic ether compound or a derivative thereof, a deprotonating agent, a ligand, and a catalyst in an organic solvent, mixing, and performing a reflux reaction; and after the reaction is complete, performing post-treatment to obtain the near-infrared emissive aggregation-induced emission organic photothermal molecule.

In an embodiment, in the step 3, the halogenated aromatic hydrocarbon or halogenated aromatic ether compound or the derivative thereof includes one or more of p-bromoanisole, 4-n-hexyloxybromobenzene, 1-bromo-4-(octadecyloxy)benzene, 1-bromo-4-hexylbenzene, 1-bromo-4-(perfluorohexyl)benzene.

In an embodiment, the deprotonating agent is sodium tert-butoxide or potassium tert-butoxide; and/or,

the ligand is 2-dicyclohexylphosphino-2,4,6-triisopropylbiphenyl or 2-dicyclohexylphosphino-2′,6′-diisopropoxy-1,1′-biphenyl; and/or, the catalyst is tris(2-benzylideneacetone) dipalladium (0).

An application of the near-infrared emissive aggregation-induced emission organic photothermal molecule in preparing near-infrared aggregation-induced emission multifunctional protein nanoparticles.

A preparation method of near-infrared aggregation-induced emission multifunctional protein nanoparticles, including the following steps:
mixing a solution of the near-infrared emissive aggregation-induced emission organic photothermal molecule with a transferrin solution to perform a self-assembly reaction; after the reaction is complete, washing, centrifuging, filtering, and collecting to obtain the near-infrared aggregation-induced emission multifunctional protein nanoparticles.

In an embodiment, the transferrin is one or more of holo-transferrin, transferrin functionally modified protein, or transferrin controllably modified protein.

In an embodiment, the concentration of the solution of the near-infrared emissive aggregation-induced emission organic photothermal molecule is 1 milligram per milliliter (mg/mL); and/or,
the concentration of the transferrin solution is 3 mg/mL; and/or,
a volume ratio/mass ratio of the solution of the near-infrared emissive aggregation-induced emission organic photothermal molecule to the transferrin solution is 1:3 (weight per weight) (w/w).

Beneficial effects are as follows. Holo-transferrin is an iron-rich protein mainly used for transporting iron absorbed from the digestive tract and iron released by red blood cell degradation. The nanoparticles encapsulated by holo-transferrin have excellent characteristics such as high stability, biocompatibility, and biodegradability, and possess a high binding capacity with organic small molecules. Transferrin is essential for ferroptosis, as it transports Fe³⁺ into endosomes of the nucleus, where Fe³⁺ is further reduced to Fe²⁺. Finally, mediated by divalent metal transporter 1, Fe²⁺ is released from the endosomes into the labile iron pool in the cytoplasm. Abnormal expression or dysfunction of these iron-related proteins will cause the intracellular iron ion concentration to increase due to metabolic imbalance, thereby triggering the Fenton reaction, leading to lipid peroxidation (LPO) and ferroptosis of brain tumor cells. Therefore, holo-transferrin, as a potential nanocarrier, may effectively cross the blood-brain barrier and simultaneously induce ferroptosis in brain tumor cells. The near-infrared emissive aggregation-induced emission organic photothermal molecule provided by the present disclosure has the characteristics of near-infrared-II luminescence, heat generation, and good photostability. Subsequently, holo-transferrin is used as the biofunctional carrier.

The present disclosure mixes the near-infrared emissive aggregation-induced emission organic photothermal molecule with holo-transferrin at a fixed ratio, uniformly loading the organic photothermal molecule onto the protein to form near-infrared molecule-holo-transferrin composite nanoparticles. There is a strong hydrogen bonding and van der Waals interaction between the organic photothermal molecule and transferrin. This ingenious and unique interaction stabilizes the performance of the protein nanoparticles. The presence of holo-transferrin enables the protein to efficiently cross the blood-brain barrier and promote the occurrence of ferroptosis in the tumor site. Moreover, the near-infrared emissive aggregation-induced emission organic photothermal molecule exhibits excellent near-infrared imaging effects and photothermal capability under laser irradiation. The obtained near-infrared aggregation-induced emission multifunctional protein nanoparticles have excellent photothermal effects and imaging resolution, exhibit excellent targeting towards brain tumors, and have practical medical significance in crossing the blood-brain barrier, tumor killing, and combined photothermal and ferroptosis therapy. The obtained near-infrared aggregation-induced emission multifunctional protein nanoparticles are expected to be widely used in intelligent medicine delivery and nanomedicine.

A near-infrared aggregation-induced emission multifunctional protein nanoparticles prepared by the preparation method.

Beneficial effects are as follows. The product structure provided by the present disclosure contains a large planar benzene ring structure with high absorption and a twisted skeleton for enhancing red-shifted emission. D-T-A structural modification and the introduction of phenyl groups improve the brightness of such aggregation-induced emission materials. The introduction of alkyl chains at the terminal of the electron donor balances the luminescence and heat generation capabilities of the material, enabling this novel material to possess both good luminescence imaging performance. The present disclosure provides a near-infrared nanotheranostic system integrating blood-brain barrier crossing, targeting, ferroptosis induction, fluorescence imaging, and photothermal therapy, possessing important prospects for scientific research and clinical application in the fields of biotechnology and medical technology.

In an embodiment, the protein carrier is spherical, polyhedral, or aggregated blocky.

The size of the near-infrared aggregation-induced emission multifunctional protein nanoparticles is 80-200 nanometers (nm).

Beneficial effects are as follows. A protein particle size of less than 200 nanometers may enhance the enhanced permeability and retention (EPR) effect of the near-infrared aggregation-induced emission multifunctional protein nanoparticles, thereby improving the effect of targeted tumor accumulation.

An application of the near-infrared aggregation-induced emission multifunctional protein nanoparticles in preparing a medicine for diagnosing or treating brain tumors.

Compared with the prior art, the present disclosure has the following advantages and technical effects.

The synthesis method for the near-infrared emissive aggregation-induced emission organic photothermal molecule provided by the present disclosure is simple and efficient. The obtained novel organic molecule possesses near-infrared-II luminescence performance and excellent photothermal performance. Meanwhile, the preparation process for the near-infrared aggregation-induced emission multifunctional protein nanoparticles in the present disclosure is simple, and the performance is excellent. The near-infrared aggregation-induced emission multifunctional protein nanoparticles may effectively cross the blood-brain barrier and accumulate in the brain, enabling visualization of brain tumors and a photothermal therapy system. There is a strong hydrogen bonding and van der Waals interaction between the molecular structure of the organic material and holo-transferrin. This ingenious and unique interaction stabilizes the performance of the protein nanoparticles.

### BRIEF DESCRIPTION OF THE DRAWINGS

The drawings constituting a part of the present disclosure are used to provide a further understanding of the present disclosure. The schematic embodiments and their descriptions of the present disclosure are used to explain the present disclosure and do not constitute an improper limitation of the present disclosure. In the drawings:

FIG. 1 is PL emission intensity and maximum emission wavelength of a near-infrared emissive aggregation-induced emission organic photothermal molecule NDA-18 obtained in Embodiment 3 tested in a mixed solvent of water and tetrahydrofuran, showing molecular emission in a near-infrared region.

FIG. 2 shows molecular docking and molecular dynamics simulation results of an interaction between a near-infrared organic molecule and holo-transferrin in near-infrared aggregation-induced emission multifunctional protein nanoparticles obtained in Embodiment 4.

FIG. 3 is a scanning electron microscope image of the near-infrared aggregation-induced emission multifunctional protein nanoparticles obtained in Embodiment 4.

FIG. 4 is a transmission electron microscope image of the near-infrared aggregation-induced emission multifunctional protein nanoparticles obtained in Embodiment 4.

FIG. 5 shows particle size distribution of the near-infrared aggregation-induced emission multifunctional protein nanoparticles obtained in Embodiment 4.

FIG. 6A shows an in vitro dialysis chamber model.

FIG. 6B shows an in vitro simulation of crossing a blood-brain barrier by the near-infrared aggregation-induced emission multifunctional protein nanoparticles obtained in Embodiment 4.

FIG. 7 shows induction of ferroptosis in U87 cells by the near-infrared aggregation-induced emission multifunctional protein nanoparticles obtained in Embodiment 4.

FIG. 8 shows a use of the near-infrared aggregation-induced emission multifunctional protein nanoparticles obtained in Embodiment 4 for tumor-targeted imaging.

FIG. 9 shows a photothermal therapy/imaging effect of the near-infrared aggregation-induced emission multifunctional protein nanoparticles obtained in Embodiment 4 on brain tumors.

FIG. 10 shows a combined therapeutic effect of the near-infrared aggregation-induced emission multifunctional protein nanoparticles obtained in Embodiment 4 on brain tumors.

FIG. 11 is a flowchart of a preparation method of the near-infrared emissive aggregation-induced emission organic photothermal molecule.

### DETAILED DESCRIPTION OF THE EMBODIMENTS

In the following, the technical solutions in the embodiments of the present disclosure will be clearly and completely described with reference to the attached drawings. Apparently, the described embodiments are only a part of the embodiments of the present disclosure, but not all the embodiments. Based on the embodiments in the present disclosure, all other embodiments obtained by one of ordinary skill in the art without creative effort belong to the protection scope of the present disclosure.

To make the above objects, features and advantages of the present disclosure more obvious and easier to understand, the present disclosure will be further described in detail with the attached drawings and specific embodiments.

Unless otherwise specified, room temperature or normal temperature in the embodiments of the present disclosure refers to 25±3 degrees Celsius (° C.).

Unless otherwise specified, the raw materials in the embodiments of the present disclosure are all purchased commercially.

Redistilled N,N-Dimethylformamide was purchased from Energy Chemical Company.

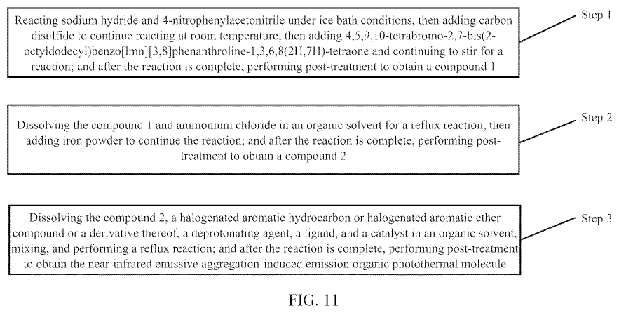

A preparation method of the near-infrared emissive aggregation-induced emission organic photothermal molecule, including the following steps, as shown in FIG. 11:
step 1 reacting sodium hydride and 4-nitrophenylacetonitrile under ice bath conditions, then adding carbon disulfide to continue the reaction at room temperature, then adding 4,5,9,10-tetrabromo-2,7-bis(2-octyldodecyl)benzo[lmn][3,8]phenanthroline-1,3,6,8 (2H,7H)-tetraone and continuing to stir for a reaction; after the reaction is complete, performing post-treatment to obtain a compound 1;
step 2 dissolving the compound 1 and ammonium chloride in an organic solvent for reflux reaction, then adding iron powder to continue the reaction; after the reaction is complete, performing post-treatment to obtain a compound 2; and
step 3 dissolving the compound 2, a halogenated aromatic hydrocarbon or halogenated aromatic ether compound or a derivative thereof, a deprotonating agent, a ligand, and a catalyst in an organic solvent, mixing, and performing a reflux reaction; and after the reaction is complete, performing post-treatment to obtain the near-infrared emissive aggregation-induced emission organic photothermal molecule.

### Embodiment 1

According to a preparation method of compound 1, the synthetic route is as follows:

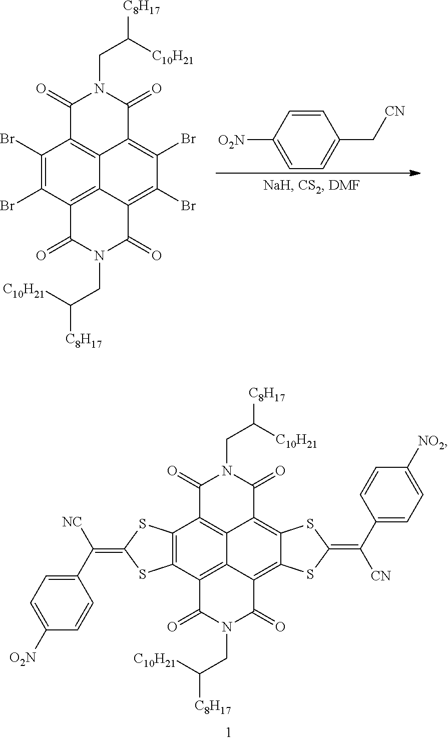

specifically including the following steps:
under nitrogen protection, adding sodium hydride (210 milligrams (mg), 5.24 millimolar (mmol)) to 10 milliliters (mL) of redistilled N,N-Dimethylformamide, and placing under ice bath conditions; then adding 4-nitrophenylacetonitrile (425 mg, 2.62 mmol) and stirring for 30 minutes; then adding carbon disulfide (300 mg, 3.93 mmol) and raising the reaction temperature to room temperature to continue the reaction for 2 hours; after the reaction solution turning from colorless to light green and gradually to brown, adding 4,5,9,10-tetrabromo-2,7-bis(2-octyldodecyl)benzo[lmn][3,8]phenanthroline-1,3,6,8 (2H,7H)-tetraone (300 mg, 0.262 mmol) at one time, and stirring at room temperature for 1 hour until the reaction solution turns black-purple; then adding 20 mL of saline to terminate the reaction, extracting with ethyl acetate (EA), collecting the organic phase, drying with anhydrous sodium sulfate, and evaporating to remove the solvent; and separating the resulting residue by column chromatography using dichloromethane and petroleum ether (4:1) as eluent to obtain a dark green solid compound 1 with a yield of 52%.

¹H NMR (500 MHz, CDCl₃), δ (ppm): 8.41 (d, J=5.0 Hz, 4H), 7.92 (d, J=10.0 Hz, 4H), 4.20-4.10 (m, 4H), 1.99 (s, 2H), 1.27 (t, 64H), 0.85 (m, 12H). ¹³C NMR (126 MHz, CDCl₃) δ [ppm]: 162.07, 147.84, 146.50, 145.96, 139.18, 128.27, 124.79, 116.03, 99.68, 46.22, 36.42, 31.94, 31.90, 30.08, 29.70, 29.66, 29.62, 29.57, 29.38, 29.35, 22.70, 22.69, 14.14. MS (MALDI-TOF) [m/z]: calcd for C₇₂H₉₀N₆O₈S₄, 1295.5737; found, 1295.5784.

### Embodiment 2

According to a preparation method of compound 2, the synthetic route is as follows:

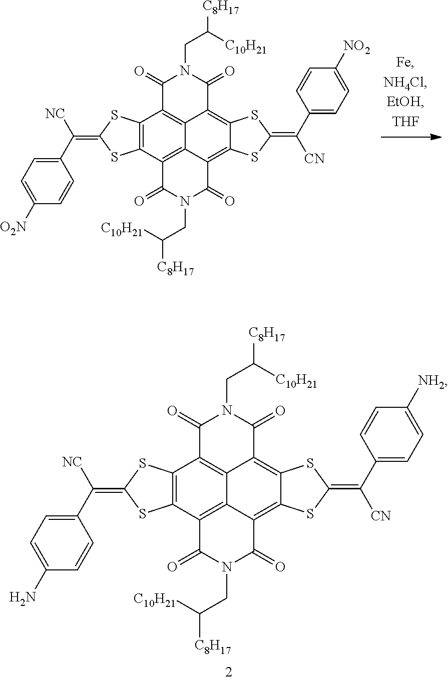

specifically including the following steps:
under nitrogen protection, dissolving 200 mg of compound 1 obtained in Embodiment 1 and 70 mg of ammonium chloride in a mixed solvent of 15 mL tetrahydrofuran and 20 mL ethanol, and refluxing at 80° C. for 10 minutes; then adding 50 mg of iron powder and continuing the reaction for 40 minutes; after the reaction is complete, cooling to room temperature, removing the solvent under reduced pressure; purifying the residue by silica gel column chromatography using a mixed solution of dichloromethane/methanol (volume ratio 10:1) as eluent to obtain the compound 2 as a green powder product with a yield of 76%.

¹H NMR (500 MHz, CDCl₃) δ [ppm]: 7.54-7.39 (d, 4H), 6.86-6.69 (d, 4H), 4.23-4.10 (d, 4H), 3.99 (s, 2H), 2.00 (s, 2H), 1.27 (m, 64H), 0.85 (dt, 12H). ¹³C NMR (126 MHz, CDCl₃) δ [ppm]: 13C NMR (126 MHz, THF) δ 162.29, 150.86, 150.04, 147.36, 144.89, 127.99, 125.22, 120.88, 117.34, 115.16, 114.12, 101.80, 45.68, 36.45, 32.09, 30.47, 29.90, 26.41, 22.00, 13.54. MS (MALDI-TOF) [m/z]: calcd for C₇₂H₉₄N₆O₄S₄, 1235.6253; found, 1235.6283.

### Embodiment 3

According to a preparation method of a near-infrared emissive aggregation-induced emission organic photothermal molecule NDA-18, the synthetic route is as follows:

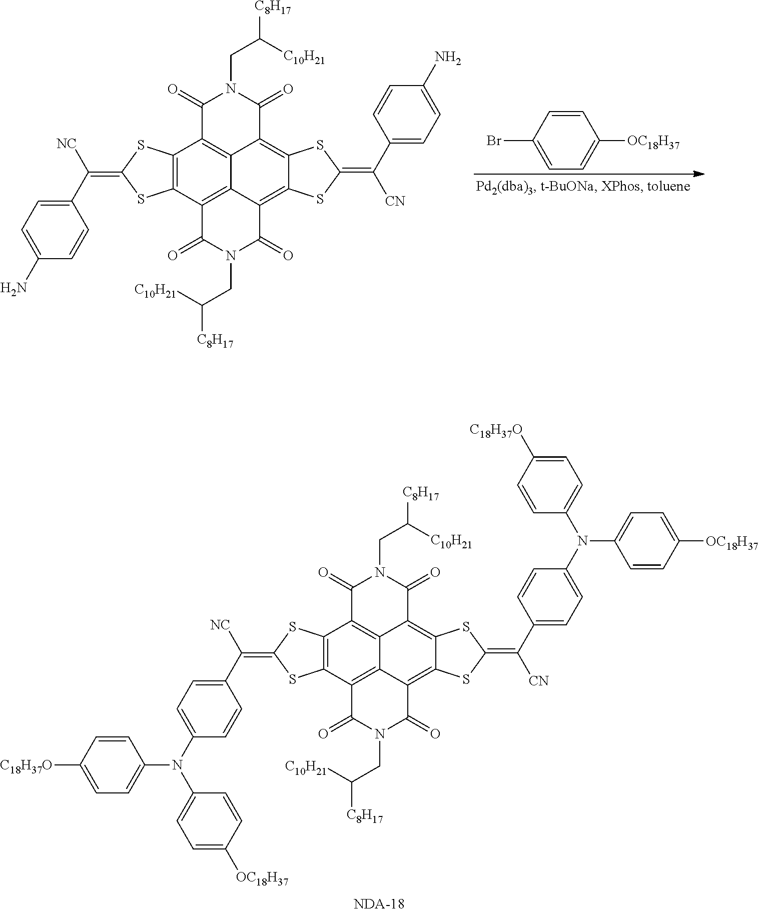

specifically including the following steps:
under nitrogen protection, dissolving compound 2 obtained in Embodiment 2 (100 mg, 0.08 mmol), 1-bromo-4-(octadecyloxy)benzene (170 mg, 0.40 mmol), sodium tert-butoxide (53.6 mg, 0.56 mmol), 2-dicyclohexylphosphino-2,4,6-triisopropylbiphenyl (18.2 mg, 0.2 mmol), tris(2-benzylideneacetone) dipalladium (0) (9.2 mg, 0.1 mmol) in toluene, and refluxing at 120° C. for 12 hours; after the reaction is complete, cooling to room temperature, extracting the mixture with dichloromethane, and washing three times with brine; then, drying the mixture over anhydrous sodium sulfate, removing the solvent under reduced pressure; purifying the residue by silica gel column chromatography using dichloromethane and petroleum ether (2:1) as eluent to obtain the near-infrared emissive aggregation-induced emission organic photothermal molecule NDA-18 as a green powder product, with a yield of 75%.

¹H NMR (500 MHz, CDCl₃) δ [ppm]: 7.49-7.44 (d, 4H), 7.18-7.12 (d, 8H), 6.99-6.94 (d, 4H), 6.94-6.86 (d, 8H), 4.24-4.16 (d, 4H), 4.00-3.96 (m, 8H), 2.05 (s, 2H), 1.27 (m, 192H), 0.85 (dt, 24H). ¹³C NMR (126 MHz, CDCl₃) δ [ppm]: 162.33, 156.40, 149.46, 139.37, 133.98, 128.05, 127.61, 124.01, 118.49, 115.50, 115.19, 115.10, 77.29, 77.04, 76.78, 68.45, 68.31, 34.89, 31.96, 31.90, 31.46, 30.21, 30.13, 29.74, 29.69, 29.66, 29.50, 29.40, 29.35, 29.00, 26.44, 26.15, 25.95, 22.72, 14.15. MS (MALDI-TOF) [m/z]: calcd for C₁₆₈H₂₅₄N₆O₈S₄, 2613.8603; found, 2613.7075.

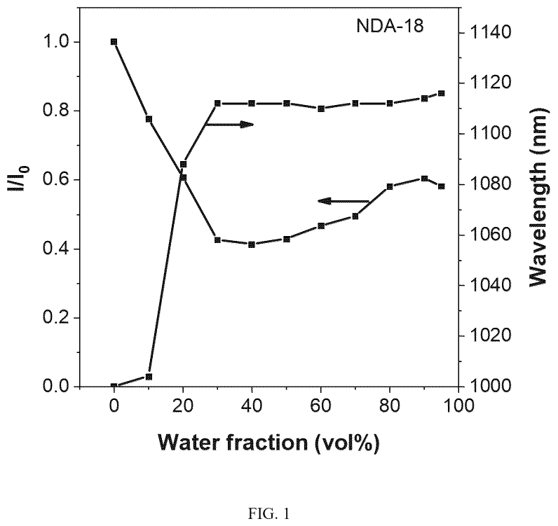

FIG. 1 is the PL intensity and maximum emission wavelength of NDA-18 in THF/water mixtures. The compound emits in the near-infrared region. Notably, the PL intensity initially decreases and then increases with increasing water content, which is characteristic of the twisted intramolecular charge transfer (TICT) mechanism and demonstrates a typical aggregation-induced emission (AIE) phenomenon.

### Embodiment 4

A preparation method of near-infrared aggregation-induced emission multifunctional protein nanoparticles, including the following steps:
dissolving 12 mg of holo-transferrin in 4 mL of ultrapure water to obtain a holo-transferrin solution, dissolving 4 mg of the near-infrared emissive aggregation-induced emission organic photothermal molecule NDA-18 obtained in Embodiment 3 in 4 mL of tetrahydrofuran to form a solution; then mixing the holo-transferrin solution with the solution of the near-infrared emissive aggregation-induced emission organic photothermal molecule uniformly at room temperature and reacting for 3 hours; after the reaction is complete, washing, centrifuging, filtering, and collecting to obtain the near-infrared aggregation-induced emission multifunctional protein nanoparticles.

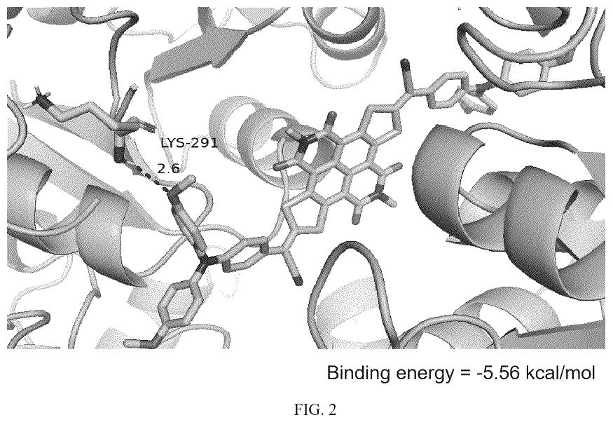

FIG. 2 shows the molecular docking and molecular dynamics simulation results of the interaction between the near-infrared organic molecule and holo-transferrin in the obtained infrared aggregation-induced emission multifunctional protein nanoparticles. It may be seen that holo-transferrin may bind with the near-infrared emissive aggregation-induced emission organic photothermal molecule at specific sites, with a binding energy of −5.56 kilocalorie per mole (kcal/mol). This proves that there is a strong hydrogen bonding and van der Waals interaction between the two, thereby stabilizing the structure and performance of the near-infrared aggregation-induced emission multifunctional protein nanoparticles.

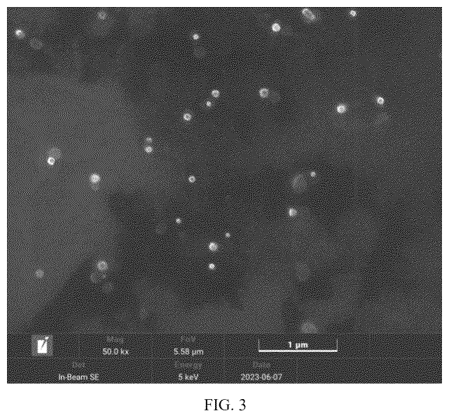

FIG. 3 is a scanning electron microscope image of the obtained near-infrared aggregation-induced emission multifunctional protein nanoparticles. It may be seen that the particle size and morphology of the near-infrared aggregation-induced emission multifunctional protein nanoparticles are uniform.

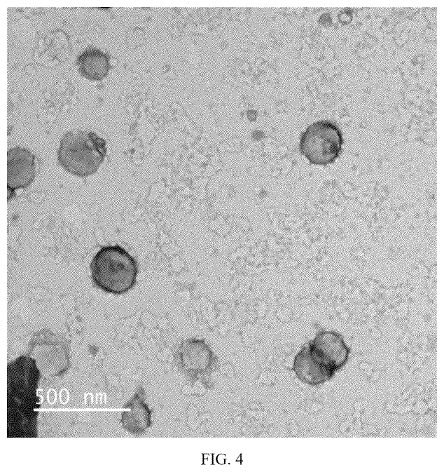

FIG. 4 is a transmission electron microscope image of the obtained near-infrared aggregation-induced emission multifunctional protein nanoparticles. It may be seen that the near-infrared aggregation-induced emission multifunctional protein nanoparticles have a particle size of about 140 nanometers and a uniform morphology.

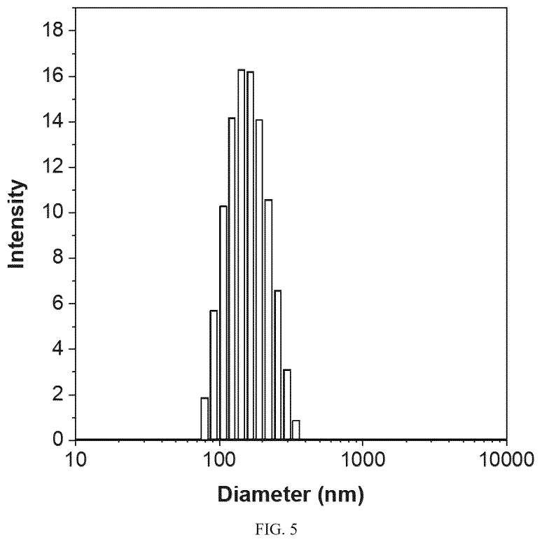

FIG. 5 shows particle size distribution of the obtained near-infrared aggregation-induced emission multifunctional protein nanoparticles. It may be seen that the near-infrared aggregation-induced emission multifunctional protein nanoparticles have a particle size of about 140 nanometers and uniform size distribution.

### Embodiment 5

A preparation method of a near-infrared emissive aggregation-induced emission organic photothermal molecule NDA-1, differing from Embodiment 3 in that 1-bromo-4-(octadecyloxy)benzene is replaced with an equimolar mass of p-bromoanisole. Other process steps and parameters are the same as in Embodiment 3. The yield is 46%. The synthetic route is as follows:

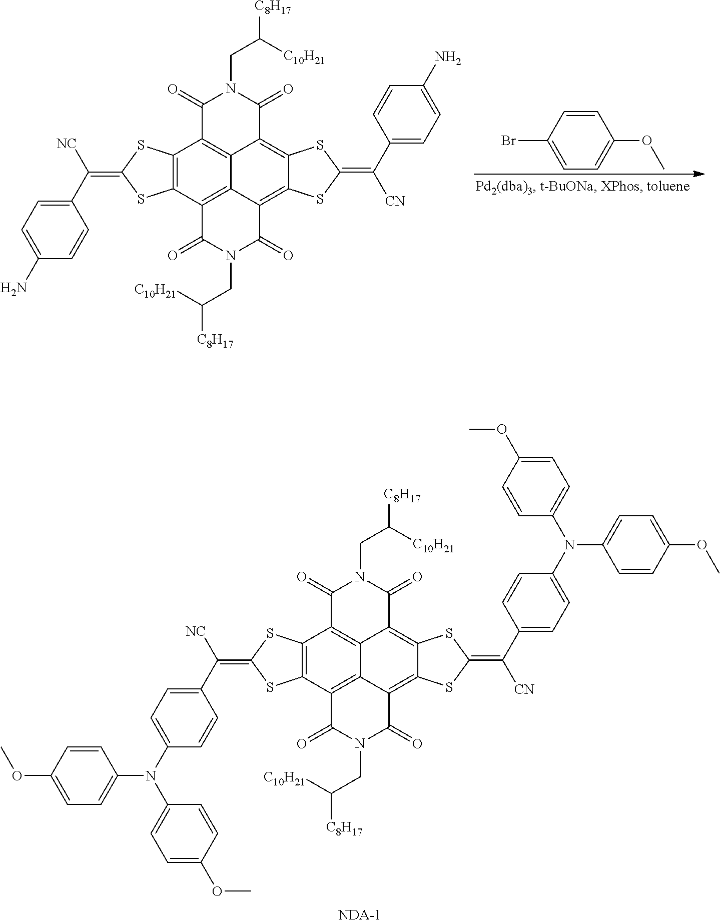

¹H NMR (500 MHz, CDCl₃) δ [ppm]: 7.54-7.39 (d, 4H), 6.86-6.69 (d, 4H), 4.23-4.10 (d, 4H), 3.99 (s, 2H), 2.00 (s, 2H), 1.27 (m, 64H), 0.85 (dt, 12H).

### Embodiment 6

A preparation method of a near-infrared emissive aggregation-induced emission organic photothermal molecule NDA-6, differing from Embodiment 3 in that 1-bromo-4-(octadecyloxy)benzene is replaced with an equimolar mass of 4-n-hexyloxybromobenzene. Other process steps and parameters are the same as in Embodiment 3. The yield is 52%. The synthetic route is as follows:

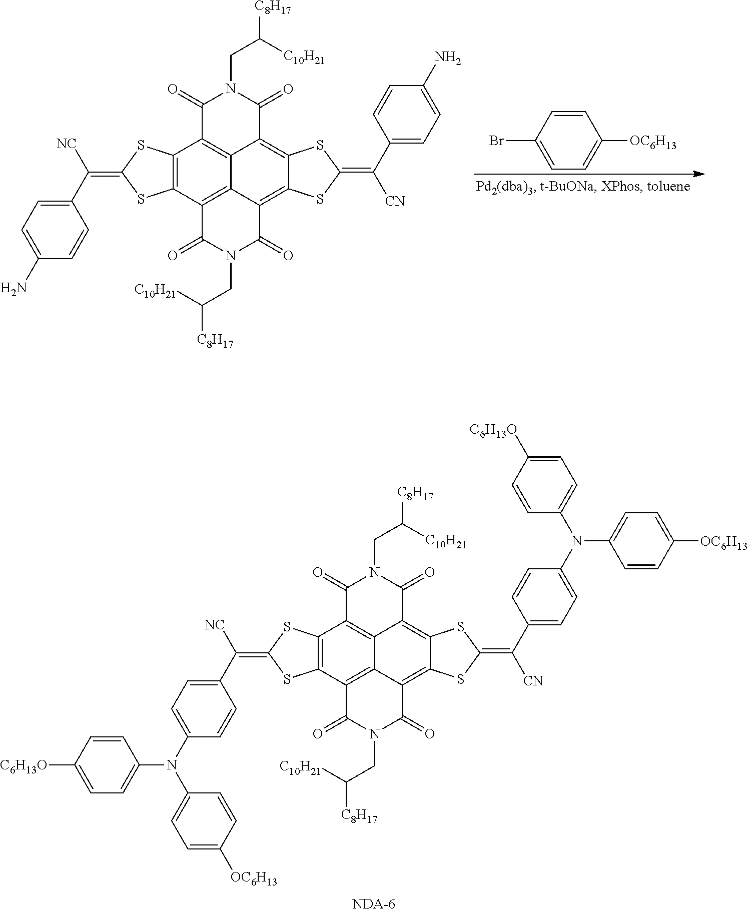

¹H NMR (500 MHz, CDCl₃) δ [ppm]: 7.49-7.44 (d, 4H), 7.18-7.12 (d, 8H), 6.99-6.94 (d, 4H), 6.94-6.86 (d, 8H), 4.24-4.16 (d, 4H), 4.00-3.96 (m, 8H), 2.05 (s, 2H), 1.27 (m, 64H), 0.85 (dt, 12H). ¹³C NMR (126 MHz, CDCl₃) δ [ppm]: 162.19, 156.44, 149.39, 139.32, 129.46, 127.95, 127.68, 127.34, 118.37, 115.52, 115.31, 114.99, 77.32, 77.07, 76.82, 68.55, 68.30, 31.96, 31.94, 31.67, 31.51, 31.40, 30.20, 29.75, 29.69, 29.40, 26.46, 25.83, 25.79, 22.73, 22.68, 22.66, 14.16, 14.09. MS (MALDI-TOF) [m/z]: calcd for C₁₂₀H₁₅₈N₆O₈S₄, 1940.1058; found, 1940.1410.

### Embodiment 7

A preparation method of a near-infrared emissive aggregation-induced emission organic photothermal molecule NDA-6A, differing from Embodiment 3 in that 1-bromo-4-(octadecyloxy)benzene is replaced with an equimolar mass of 1-bromo-4-hexylbenzene. Other process steps and parameters are the same as in Embodiment 3. The yield is 61%. The synthetic route is as follows:

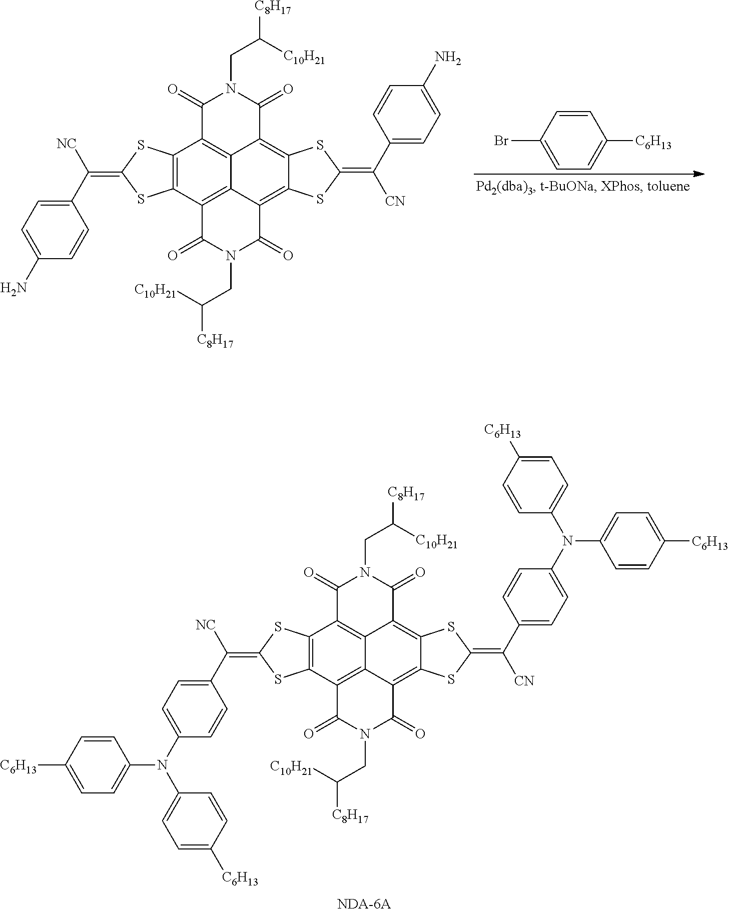

¹H NMR (500 MHz, CDCl₃) δ [ppm]: 7.53-7.02 (m, 24H), 4.19 (s, 4H), 2.60 (s, 8H), 2.03 (s, 2H), 1.27 (m, 64H), 0.84 (d, 12H). ¹³C NMR (126 MHz, CDCl₃) δ [ppm]: 144.24, 143.38, 139.19, 130.29, 129.49, 129.02, 128.04, 126.56, 125.80, 125.16, 124.39, 121.26, 120.23, 35.53, 34.05, 31.78, 31.52, 31.10, 30.15, 29.68, 29.38, 29.15, 25.52, 22.71, 22.67, 22.27, 14.15. MS (MALDI-TOF) [m/z]: calcd for C₁₂₀H₁₅₈N₆O₄S₄, 1876.1261; found, 1876.1603.

### Embodiment 8

A preparation method of a near-infrared emissive aggregation-induced emission organic photothermal molecule NDA-6F, differing from Embodiment 3 in that 1-bromo-4-(octadecyloxy)benzene is replaced with an equimolar mass of 1-bromo-4-(perfluorohexyl)benzene. Other process steps and parameters are the same as in Embodiment 3. The yield is 49%. The synthetic route is as follows:

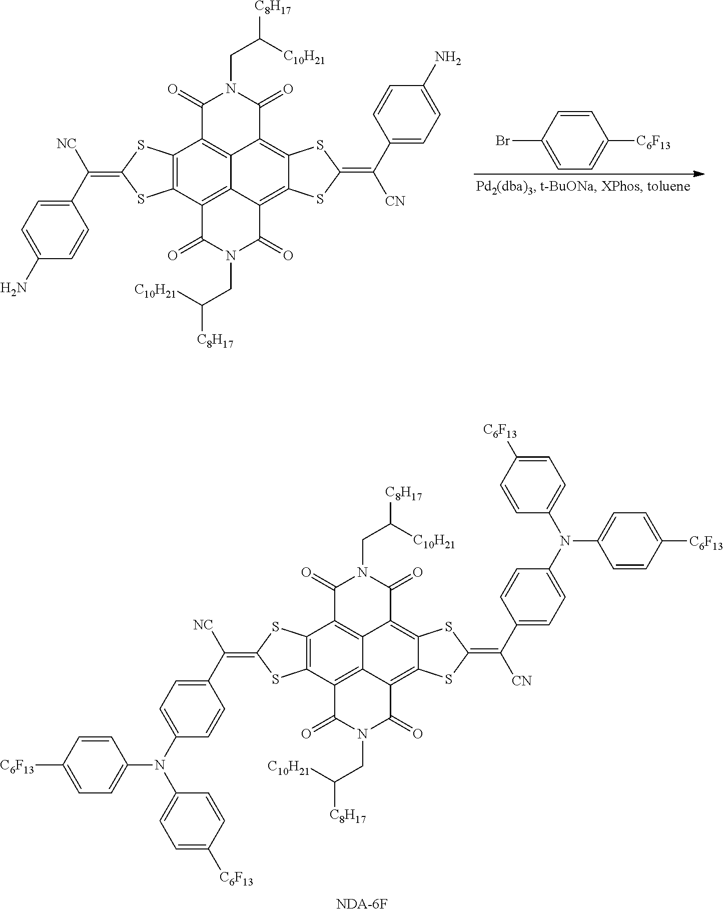

¹H NMR (500 MHz, CDCl₃) δ [ppm]: 7.70-7.50 (m, 12H), 7.35-7.22 (m, 12H), 4.21 (s, 4H), 2.07 (s, 2H), 1.22 (m, 64H), 0.85 (s, 12H). ¹⁹F NMR (500 MHz, CDCl₃) δ [ppm]: −81.30 (s, 3F), −110.70 (s, 2F), −122.02 (s, 2F), −122.13 (S, 2F), −123.35 (S, 2F), −126.67 (S, 2F). ¹³C NMR (126 MHz, CDCl₃) δ [ppm]: 162.23, 162.07, 149.51, 146.85, 144.85, 139.34, 128.74, 128.56, 125.16, 124.89, 124.13, 116.95, 115.76, 114.10, 101.01, 46.25, 36.37, 34.90, 33.87, 31.97, 31.92, 31.87, 31.82, 31.65, 31.54, 31.47, 30.33, 30.21, 30.14, 29.74, 29.71, 29.64, 29.58, 29.41, 29.38, 29.34, 29.30, 29.21, 28.99, 26.42, 24.77, 22.74, 22.71, 22.69, 22.67, 22.62, 14.17, 14.13, 14.08, 14.04, 14.00, 11.19. MS (MALDI-TOF) [m/z]: calcd for C₁₂₀H₁₀₆F₅₂N₆O₄S₄, 2811.6362; found, 2811.3159.

### Embodiment 9-Embodiment 12

A preparation method of near-infrared aggregation-induced emission multifunctional protein nanoparticles, differing from Embodiment 4 only in that the near-infrared emissive aggregation-induced emission organic photothermal molecule NDA-18 obtained in Embodiment 3 is replaced with an equal amount of the near-infrared emissive aggregation-induced emission organic photothermal molecule obtained in Embodiment 5, Embodiment 6, Embodiment 7, or Embodiment 8, respectively. Other process steps and parameters are the same as in Embodiment 4.

Technical effects are as follows.

1. In vitro simulation experiment of the near-infrared aggregation-induced emission multifunctional protein nanoparticles crossing the blood-brain barrier.

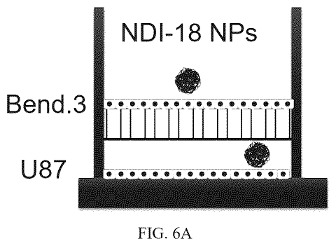

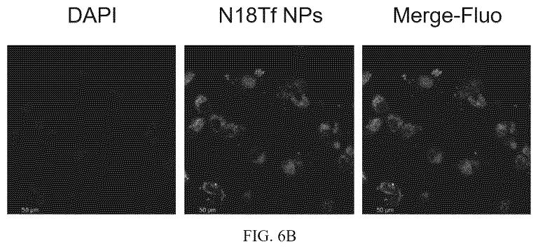

BBB permeability is evaluated in an in vitro dialysis chamber model. The upper chamber is seeded with a layer of endothelial cells brain-derived Endothelial cells.3 (bEnd.3) to simulate the blood-brain barrier, while the lower chamber is seeded with U87 cells to simulate glioblastoma tumors (FIG. 6A).

FIG. 6B shows the in vitro simulation of the near-infrared aggregation-induced emission multifunctional protein nanoparticles crossing the blood-brain barrier. It may be seen that the fluorescein isothiocyanate (FITC)-labeled near-infrared aggregation-induced emission multifunctional protein nanoparticles (labeled as N18TfNPs in the figure) show a strong signal in the cytoplasm of U87 cells in the lower chamber, proving that the FITC-labeled near-infrared aggregation-induced emission multifunctional protein nanoparticles have good in vitro blood-brain barrier penetration and cellular uptake capabilities.

2. Induction of ferroptosis in U87 cells by the near-infrared aggregation-induced emission multifunctional protein nanoparticles.

To verify that the particles induce ferroptosis in U87 cells, a Western blot experiment is designed as follows.

Firstly, U87 cells are divided into two groups. The experimental group is treated with the multifunctional protein nanoparticles prepared in Embodiment 4, while the control group is not treated. Afterwards, a specific primary antibody targeting the ferroptosis-related marker protein glutathione peroxidase (GPX) is used for incubation. Detection is performed using a secondary antibody (horseradish peroxidase-labeled (HRP-labeled)) and developed with a chemiluminescent substrate. Analysis of the expression differences of the ferroptosis-related protein between the experimental group and control group shows a decrease in GPX expression in the experimental group, indicating that the particle treatment may induce ferroptosis in U87 cells.

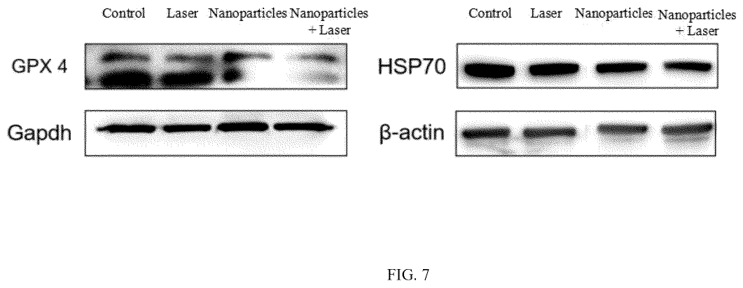

FIG. 7 shows the induction of ferroptosis in U87 cells by the near-infrared aggregation-induced emission multifunctional protein nanoparticles obtained in Embodiment 4. It may be seen that after adding the near-infrared aggregation-induced emission multifunctional protein nanoparticles (corresponding to the right two lanes of bands), the GPX4 protein is significantly reduced, indicating that the cells may be undergoing oxidative stress and an iron-related death pathway.

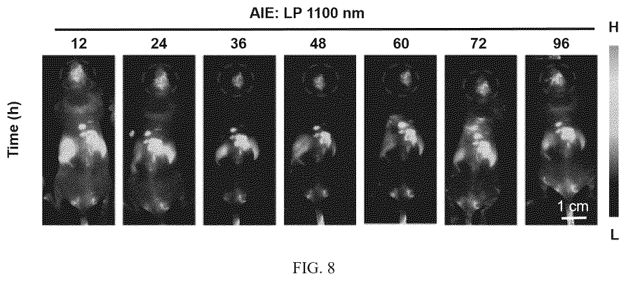

3. Tumor-targeted imaging with near-infrared aggregation-induced emission multifunctional protein nanoparticles. The results are shown in FIG. 8. It may be seen that the fluorescence signal of the nanoparticle at the tumor site gradually increases, reaching the peak at 12 hours, indicating effective accumulation and targeting of the tumor site.

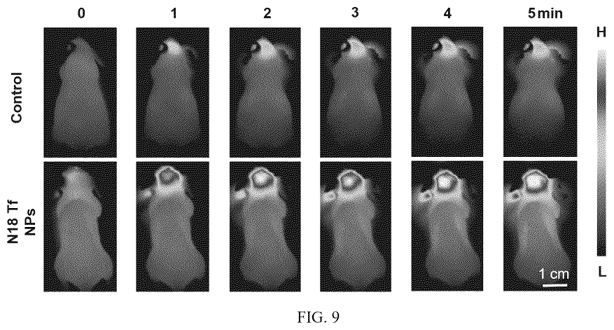

FIG. 9 shows the photothermal therapy/imaging effect of the near-infrared aggregation-induced emission multifunctional protein nanoparticles obtained in Embodiment 4 on brain tumors. It may be seen that after adding the near-infrared aggregation-induced emission multifunctional protein nanoparticles, the maximum temperature in the brain tumor region reaches 42 degrees Celsius within 5 minutes. This temperature shows a significant difference from the ambient temperature, demonstrating precise and efficient photothermal killing capability in the brain tumor region.

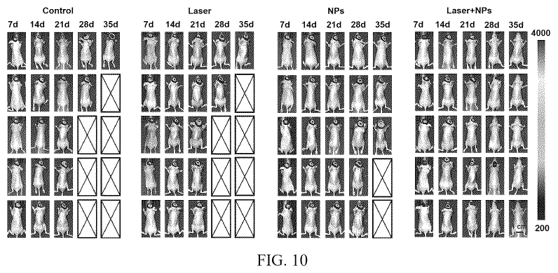

FIG. 10 shows the combined therapeutic effect of the near-infrared aggregation-induced emission multifunctional protein nanoparticles obtained in Embodiment 4 on brain tumors, specifically including the following steps:
the near-infrared aggregation-induced emission multifunctional protein nanoparticles not only have efficient tumor ferroptosis induction ability, but also have efficient photothermal capability to ensure the smooth progress of photothermal therapy after turning on the 808 laser, thus achieving a combined brain tumor treatment mode of ferroptosis/photothermal killing. According to FIG. 10, it may be seen that during the treatment period, the nanoparticle treatment group showed a longer survival rate, indicating the potential of the nanoparticles for combined therapy of brain tumors.

The above are only specific preferred embodiments of the present disclosure, but the protection scope of the present disclosure is not limited thereto. One of ordinary skill in the art familiar with the technical field may easily think of changes or substitutions within the technical scope disclosed in the present disclosure, which should be covered within the protection scope of the present disclosure. Therefore, the protection scope of the present disclosure shall be subject to the protection scope of the claims.

What is claimed is:

1. A near-infrared emissive aggregation-induced emission organic photothermal molecule, wherein a structure is shown in formula I: 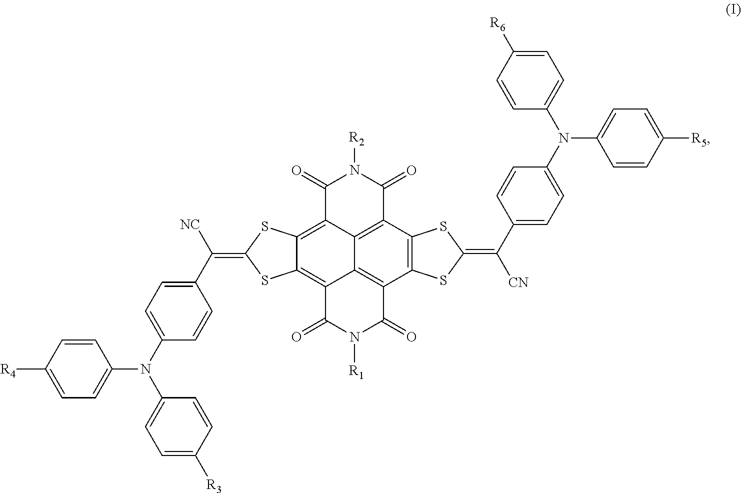
wherein R₁, R₂, R₃, R₄, R₅, R₆ are independently selected from following groups:
C₁-C₂₀ straight-chain alkyl, C₁-C₂₀ branched-chain alkyl, wherein carbon atoms may be substituted by oxygen atoms, and hydrogen atoms may be substituted by one or more of fluorine atoms, chlorine atoms, bromine atoms, or iodine atoms.

2. A near-infrared emissive aggregation-induced emission organic photothermal molecule, being selected from following structures:

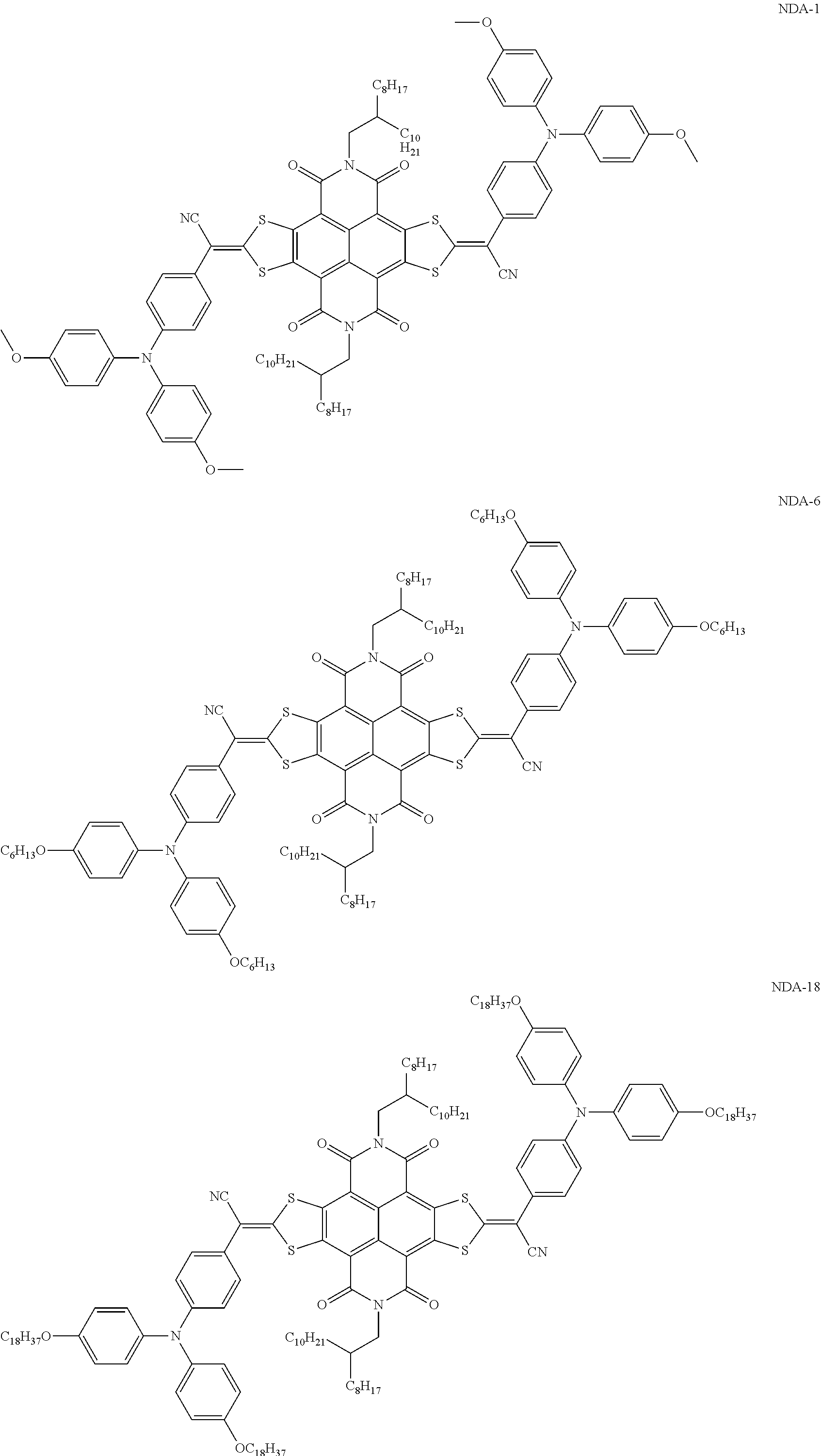

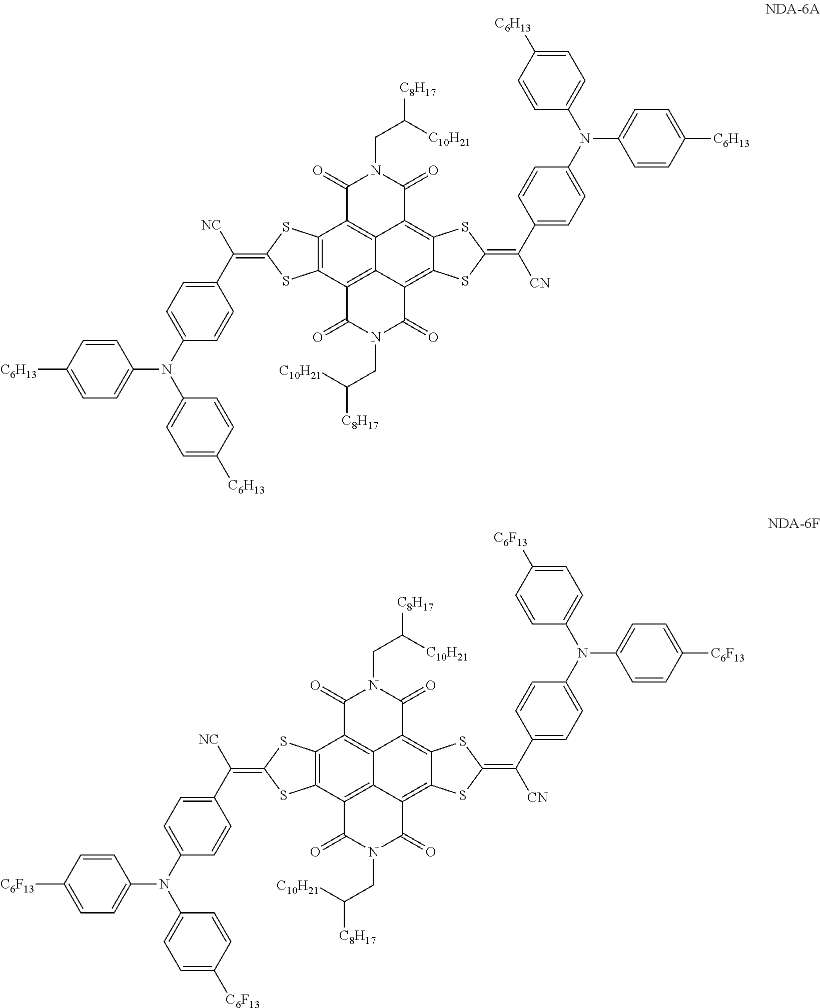

3. A preparation method of the near-infrared emissive aggregation-induced emission organic photothermal molecule according to claim 2, comprising the following steps:
(1) reacting sodium hydride and 4-nitrophenylacetonitrile under ice bath conditions, then adding carbon disulfide to continue reacting at room temperature, then adding 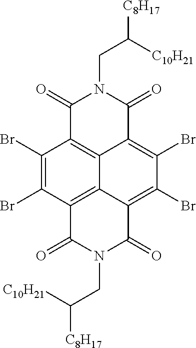
and continuing to stir for a reaction; and after the reaction is complete, performing post-treatment to obtain a compound 1 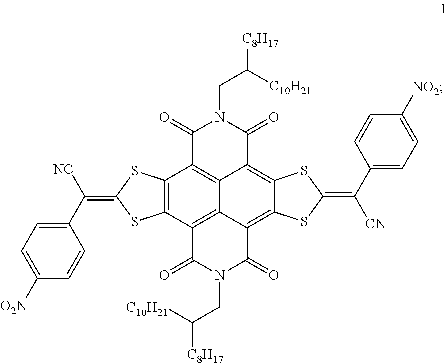
(2) dissolving the compound 1 and ammonium chloride in an organic solvent for a reflux reaction, then adding iron powder to continue the reaction; and after the reaction is complete, performing post-treatment to obtain a compound 2 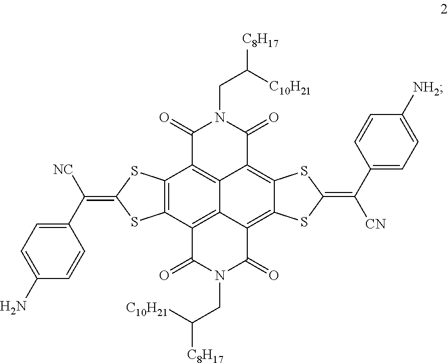
and
(3) dissolving the compound 2, a halogenated aromatic hydrocarbon or halogenated aromatic ether compound or a derivative thereof, a deprotonating agent, a ligand, and a catalyst in an organic solvent, mixing, and performing a reflux reaction; and after the reaction is complete, performing post-treatment to obtain the near-infrared emissive aggregation-induced emission organic photothermal molecule;
wherein in the step (3), the halogenated aromatic hydrocarbon or the halogenated aromatic ether compound or the derivative thereof is selected from one or more of p-bromoanisole, 4-n-hexyloxybromobenzene, 1-bromo-4-(octadecyloxy)benzene, 1-bromo-4-hexylbenzene, or 1-bromo-4-(perfluorohexyl)benzene.

4. The preparation method of the near-infrared emissive aggregation-induced emission organic photothermal molecule according to claim 3, wherein the deprotonating agent is sodium tert-butoxide or potassium tert-butoxide.

5. The preparation method of the near-infrared emissive aggregation-induced emission organic photothermal molecule according to claim 3, wherein the ligand is 2-dicyclohexylphosphino-2′,4′,6′-triisopropylbiphenyl or 2-dicyclohexylphosphino-2′,6′-diisopropoxy-1,1′-biphenyl.

6. The preparation method of the near-infrared emissive aggregation-induced emission organic photothermal molecule according to claim 3, wherein the catalyst is tris(2-benzylideneacetone) dipalladium (0).

7. A preparation method of near-infrared aggregation-induced emission multifunctional protein nanoparticles, comprising the following steps:
mixing a solution of the near-infrared emissive aggregation-induced emission organic photothermal molecule according to claim 1 with a transferrin solution to perform a self-assembly reaction; after the self-assembly reaction is complete, washing, centrifuging, filtering, and collecting to obtain the near-infrared aggregation-induced emission multifunctional protein nanoparticles.

8. The preparation method of the near-infrared aggregation-induced emission multifunctional protein nanoparticles according to claim 7, wherein the transferrin is one or more of holo-transferrin, or transferrin functionally modified protein.

9. A near-infrared aggregation-induced emission multifunctional protein nanoparticle prepared by the preparation method according to claim 7.
